(19) United States
(12) Patent Application Publication
(10) Pub. No.: US 2021/0045828 A1
(43) Pub. Date: Feb. 18, 2021

(54) SURGICAL DRAPE

(71) Applicant: CMR SURGICAL LIMITED, Cambridge (GB)

(72) Inventors: Martin McBrien, Cambridge, Cambridgeshire (GB); Aidan Hird, Cambridge, Cambridgeshire (GB); Keith Marshall, Cambridge, Cambridgeshire (GB)

(21) Appl. No.: 16/966,240

(22) PCT Filed: Jan. 30, 2019
(86) PCT No.: PCT/GB2019/050262

§ 371 Date: Jul. 30, 2020

(30) Foreign Application Priority Data
Jan. 30, 2018 (GB) 1801506.5

(51) Int. Cl. A61B 46/10; A61B 34/30; A61B 46/00

(52) U.S. Cl. CPC A61B 46/10; A61B 46/40; A61B 34/30

(57) ABSTRACT

A surgical robot drape for enveloping a joint of a surgical robot, the joint configured to permit axial rotation of a part of a robot arm adjacent the joint about a joint axis, the drape comprising a proximal drape material portion configured to provide a sterile barrier over, and to be rotationally fast with, a part of the robot proximal of the joint, and a distal drape material portion configured to provide a sterile barrier over, and to be rotationally fast with, a part of the robot distal of the joint; the drape being configured such that the proximal and distal drape material portions are freely rotatable relative to one another about the joint axis.

The present disclosure relates to surgical drapes, in particular to surgical robot drapes, for example surgical robot drapes that can accommodate movement about a robot arm joint.

### BACKGROUND

It is known to use robots for assisting and performing surgery. A surgical robot typically comprises a base on which a robot arm can be supported. An instrument couples to the end of the robot arm distal of the base and supports an end effector for engaging in a medical procedure. The robot arm comprises multiple flexible joints along its length, which are used to locate the surgical instrument in a desired location relative to a patient. The surgical instrument can penetrate the body of the patient at a port so as to access a surgical site. The end effector can be used to perform the medical procedure at the surgical site.

The surgical instrument needs to be sterile. The robot arm need not be sterile. Typically, a surgical drape is provided for covering the robot arm. The surgical drape provides a sterile barrier between the surgical instrument and the robot arm. The drape provides a boundary between the robot arm and the sterile field in which the robot arm is positioned, for example an operating theatre.

The drape can be provided in the form of a large loose sheet of an impermeable membrane. Covering the robot arm with such a large loose sheet can mean that the sheet is loose about the robot arm and can interfere with the procedure being carried out.

There is a need for an improved surgical drape.

### SUMMARY

According to an aspect of the invention there is provided a surgical robot drape for enveloping a joint of a surgical robot, the joint configured to permit axial rotation of a part of a robot arm adjacent the joint about a joint axis, the drape comprising: a proximal drape material portion configured to provide a sterile barrier over, and to be rotationally fast with, a part of the robot proximal of the joint, and a distal drape material portion configured to provide a sterile barrier over, and to be rotationally fast with, a part of the robot distal of the joint; the drape being configured such that the proximal and distal drape material portions are freely rotatable relative to one another about the joint axis.

The drape may be configured to restrict the passage of fluid and/or particulate matter between the proximal and distal drape material portions so as to maintain the sterile barrier. The drape may comprise a coupling arrangement for coupling the proximal drape material portion to the distal drape material portion. The coupling arrangement may be configured to sealingly couple the proximal and distal drape material portions. The coupling arrangement may be configured to permit unconstrained relative rotation between the proximal and distal drape material portions.

The coupling arrangement may comprise a first coupling and a second coupling configured to couple to one another, wherein one of the proximal drape material portion and the distal drape material portion comprises the first coupling and the other of the proximal drape material portion and the distal drape material portion comprises the second coupling. The first coupling and the second coupling may be configured to engage with one another by a snap-fit engagement.

One or both of the first coupling and the second coupling may be ring-shaped. The coupling arrangement may be configured to permit free rotation about an axis of the ring.

One of the first and second couplings may comprise a recess and the other of the first and second couplings may comprise a protrusion, the recess being configured to receive the protrusion therein. The recess may comprise an annular recess. The protrusion may be configured to snap-fit past one of a lip and a rib adjacent the recess. One or more of the protrusion, the lip and the rib may be resiliently deformable.

The first coupling may comprise a third coupling, and the first coupling may be configured to couple to the second coupling via the third coupling. The third coupling may be freely rotatable relative to the second coupling. The coupling arrangement may be configured so that the second coupling and the third coupling are configured to engage with one another by a snap-fit engagement.

The third coupling may comprise one of a recess and a protrusion for engagement with the protrusion or recess of the second coupling.

One of the proximal drape material portion and the distal drape material portion may comprise a collar and the other of the proximal drape material portion and the distal drape material portion may comprise a collar shroud. The collar shroud may be configured to be a close fit about the collar. One or both of the collar and the collar shroud may comprise a resilient portion.

The material of the respective drape material portion may comprise the resilient portion. The resilient portion may be configured to urge a circumferential extent of the respective drape material portion to adopt a relatively smaller circumference than when unconstrained.

The drape may be configured to permit unconstrained relative rotation between the proximal and distal drape material portions about the joint axis.

The drape may be configured such that there is no restriction on the number of relative rotations between the proximal and distal drape material portions.

At least one of the proximal drape material portion and the distal drape material portion may comprise a magnetic material.

The joint may comprise a roll joint.

The proximal drape material portion may comprise a cart drape portion and the distal drape material portion may comprise an arm drape portion.

According to another aspect of the invention, there is provided a surgical robotic system comprising a surgical robot and a surgical robot drape as defined herein, in which the surgical robot drape is disposed about the surgical robot so as to envelop a joint of the surgical robot.

According to another aspect of the invention, there is provided a surgical robot arm drape for providing a sterile barrier around a portion of a surgical robot arm comprising a joint, the drape comprising: a sheet configured to form an enclosure, the enclosure having first and third regions spaced apart from one another by a second region, the second region comprising a material or configuration having a lower effective modulus than the first and third regions.

The second region may be configured to deform under external force more easily than the first and third regions.

The second region may comprise a ruckle control portion. The ruckle control portion may comprise a concertina section which is configurable between an expanded state and a contracted state.

The second region may comprise a material having a higher resilience than material of the first and third regions. The second region may comprise a material which is thinner than material of the first and third regions. The second region may comprise material of the drape that is less constrained than material of the drape in the first and third regions.

The drape may be configured to be attached to a robot arm by a plurality of fixing arrangements, one fixing arrangement being provided at or towards either end of the second region.

Material of the sheet may form or be adapted to form the enclosure.

The enclosure may be configured to enclose a longitudinally extending robot arm. The enclosure may comprise a generally tubular section. The generally tubular section may comprise the first, second and third regions. The generally tubular section may comprise at least one of a generally cylindrical section and a generally frustoconical section.

The first, second and third regions may be unitarily formed.

The second region may be able to accommodate a greater flexion of a joint enveloped by the enclosure than the first and third regions.

According to another aspect of the invention, there is provided a surgical robotic system comprising a surgical robot arm drape as defined herein and a surgical robot arm having a joint, the joint of the arm being receivable within the enclosure.

The joint may be receivable within the second region of the enclosure.

According to another aspect of the invention, there is provided a surgical robotic system comprising a surgical robot arm drape as defined herein and a surgical robot arm having a joint, the enclosure being configured such that the second region aligns with the joint.

Any one or more feature of any aspect above may be combined with any one or more feature of any other aspect above. Any apparatus feature may be written as a method feature where possible, and vice versa. These have not been written out in full here merely for the sake of brevity.

This Summary is provided to introduce a selection of concepts in a simplified form that are further described below in the Detailed Description. The mention of features in this Summary does not indicate that they are key features or essential features of the invention or of the claimed subject matter, nor is it to be taken as limiting the scope of the claimed subject matter.

### BRIEF DESCRIPTION OF THE DRAWINGS

The present invention will now be described by way of example with reference to the accompanying drawings. In the drawings:

FIG. 1 illustrates an example of a surgical robot;

FIG. 2 illustrates another example of a surgical robot;

FIG. 3 illustrates an example of a surgical robot covered by a drape;

FIGS. 4a and 4b illustrate an example of a surgical robot and a drape;

FIGS. 5a and 5b illustrate another example of a surgical robot and a drape;

FIG. 6 illustrates an example of a coupling arrangement;

FIG. 7 illustrates another example of a coupling arrangement;

FIG. 8 illustrates another example of a surgical robot and a drape; and

FIG. 9 illustrates an example of part of a surgical drape.

### DETAILED DESCRIPTION

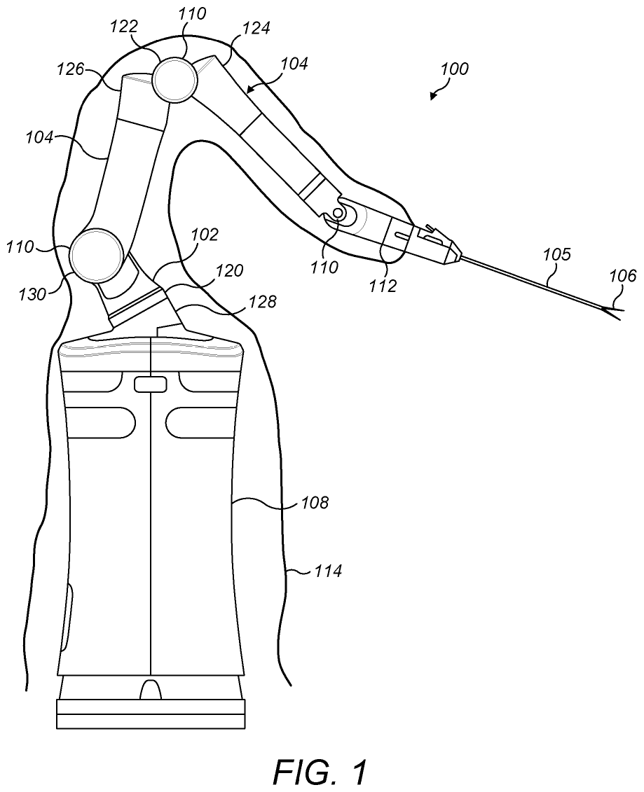

FIG. 1 illustrates a surgical robot 100 which comprises a robot arm. The robot arm comprises a base 102 of the arm and a plurality of arm segments 104. The surgical robot comprises an instrument 105 coupled to an end of the robot arm, the instrument comprising an end effector 106. The base supports the remainder of the robot arm on, for example, the operating theatre floor, the operating theatre ceiling or a trolley or cart 108. The arm extends from the base of the arm to a distal end of the arm. The instrument is coupled to the distal end of the arm. The arm is articulated by means of multiple flexible joints 110 along its length, which are used to locate the surgical instrument in a desired location relative to a patient. The joints 110 provide articulation between parts of the surgical robot attached to either side of each joint, for example two arm segments 104 or the base 102 of the arm and a mount (such as a cart or ceiling mount) to which the robot arm is mounted.

The surgical instrument 105 is attached to the distal end 112 of the robot arm. The surgical instrument can penetrate the body of the patient at a port so as to access a surgical site. At its distal end, the instrument comprises the end effector 106 for engaging in a medical procedure.

The surgical robot 100 is shrouded by a surgical drape 114 to provide a sterile boundary between the surgical instrument (which must be sterile) and the robotic arm and cart 108 (which may not be sterile). The drape provides a boundary between the robot and the sterile field in which the robot is positioned (for example an operating theatre).

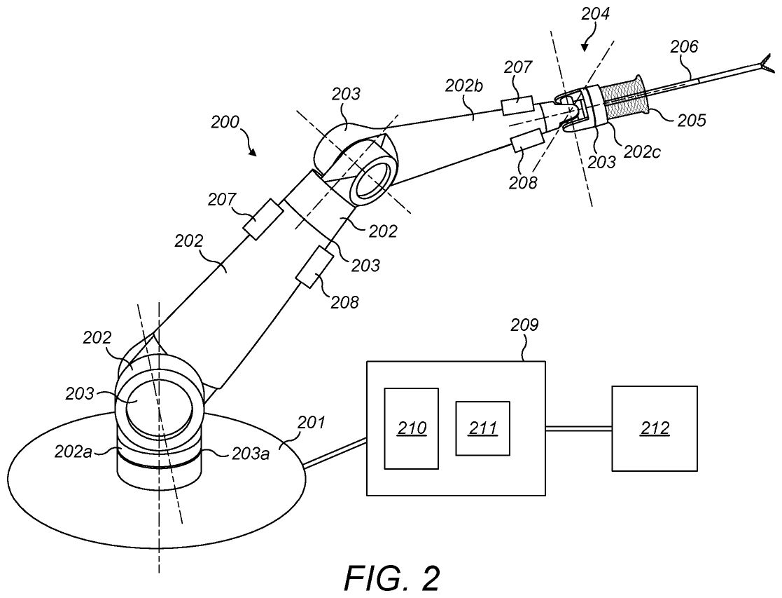

FIG. 2 illustrates a surgical robot having an arm 200 which extends from a mount 201. The mount 201 can be part of a trolley or cart, for example a mount at the top of such a cart. The arm comprises a number of rigid limbs 202. The limbs are coupled by revolute joints 203. The most proximal limb 202a is coupled to the mount by a proximal joint 203a. It and the other limbs are coupled in series by further ones of the joints 203. Suitably, a wrist 204 is made up of four individual revolute joints. The wrist 204 couples one limb (202b) to the most distal limb (202c) of the arm. The most distal limb 202c carries an attachment 205 for a surgical instrument 206. Each joint 203 of the arm has one or more motors 207 which can be operated to cause rotational motion at the respective joint, and one or more position and/or torque sensors 208 which provide information regarding the current configuration and/or load at that joint. Suitably, the motors are arranged proximally of the joints whose motion they drive, so as to improve weight distribution. For clarity, only some of the motors and sensors are shown in FIG. 2. The arm may be generally as described in our published patent application WO2015132549.

The arm terminates in the attachment 205 for interfacing with the instrument 206. An instrument interface can be mounted to a drive assembly interface of the attachment. The instrument may have a diameter less than 8 mm. Suitably, the instrument has a 5 mm diameter. The instrument may have a diameter which is less than 5 mm. The instrument diameter may be the diameter of the shaft. The instrument diameter may be the diameter of the profile of the articulation. Suitably, the diameter of the profile of the articulation matches or is narrower than the diameter of the shaft. The attachment 205 comprises a drive assembly for driving articulation of the instrument. Movable interface elements of the drive assembly interface mechanically engage corresponding movable interface elements of the instrument interface in order to transfer drive from the robot arm to the instrument. One instrument is exchanged for another several times during a typical operation. Thus, the instrument is attachable to and detachable from the robot arm during the operation.

The instrument 206 comprises an end effector for performing an operation. The end effector may take any suitable form. For example, the end effector may be smooth jaws, serrated jaws, a gripper, a pair of shears, a needle for suturing, a camera, a laser, a knife, a stapler, a cauteriser, a suctioner and so on. The instrument comprises an articulation between the instrument shaft and the end effector. The articulation comprises several joints which permit the end effector to move relative to the shaft of the instrument. The joints in the articulation are actuated by driving elements, such as cables. These driving elements are secured at the other end of the instrument shaft to the interface elements of the instrument interface. Thus, the robot arm transfers drive to the end effector as follows: movement of a drive assembly interface element moves an instrument interface element which moves a driving element which moves a joint of the articulation which moves the end effector.

Controllers for the motors, torque sensors and encoders are distributed within the robot arm. The controllers are connected via a communication bus to a control unit 209. The control unit 209 comprises a processor 210 and a memory 211. The memory 211 stores in a non-transient way software that is executable by the processor to control the operation of the motors 207 to cause the arm 200 to operate in the manner described herein. In particular, the software can control the processor 210 to cause the motors (for example via distributed controllers) to drive in dependence on inputs from the sensors 208 and from a surgeon command interface 212. The control unit 209 is coupled to the motors 207 for driving them in accordance with outputs generated by execution of the software. The control unit 209 is coupled to the sensors 208 for receiving sensed input from the sensors, and to the command interface 212 for receiving input from it. The respective couplings may, for example, each be electrical or optical cables, or may be provided by a wireless connection. The command interface 212 comprises one or more input devices whereby a user can request motion of the end effector in a desired way. The input devices could, for example, be manually operable mechanical input devices such as control handles or joysticks, or contactless input devices such as optical gesture sensors. The software stored in the memory 211 is configured to respond to those inputs and cause the joints of the arm and instrument to move accordingly, in compliance with a pre-determined control strategy. The control strategy may include safety features which moderate the motion of the arm and instrument in response to command inputs. Thus, in summary, a surgeon at the command interface 212 can control the instrument 206 to move in such a way as to perform a desired surgical procedure. The control unit 209 and/or the command interface 212 may be remote from the arm 200.

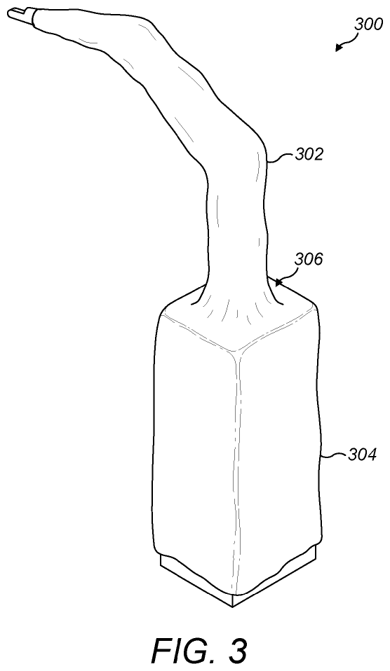

During an operation or surgical procedure, the surgical robot is shrouded in a sterile drape (an example of which is illustrated at 300 in FIG. 3) to provide a sterile barrier between the non-sterile surgical robot and the sterile operating environment. The portion of the robot that is covered by the drape then need not be sterile. The surgical instrument is sterilised before being attached to the surgical robot. The sterile drape is typically constructed of a plastic sheet, for example made of polyester, polypropylene, polyethylene or polytetrafluoroethylene (PTFE). Suitably, the drape is flexible and/or deformable. This can assist in the drape shrouding the robot arm without interfering in the location and/or movement of the robot arm and/or instruments during the surgical procedure.

In the example illustrated in FIG. 3, the drape comprises a part for draping an arm 302 and a part for draping a cart 304. The arm part of the drape and the cart part of the drape may be unitary with one another, as illustrated. To reduce the chances of the drape interfering in the location and/or movement of the robot arm and/or instruments during the surgical procedure, the drape can be of a generally close-fitting form to one or both of the robot arm and the cart. The robot arm and cart will be of known dimensions, so the drape can be configured, or manufactured, so as to closely fit about the robot arm or cart with which it is desired to use the drape.

As can be seen from the illustration in FIG. 3, the cart can be shaped substantially as a rectangular prism. The cart part of the drape suitably also adopts a similar shape so as to closely cover the cart. This means that the cart part of the drape has particular orientations at which it can be applied over the cart. As illustrated, for a rectangular prism-shaped cart, there will be four orientations, separated by 90 degrees, at which the cart part of the drape can be applied over the cart.

Once applied over the cart, the cart part of the drape will not be able to substantially twist, or rotate, with respect to the cart. The cart part of the drape will at least substantially maintain its orientation with respect to the cart. This can mean that, for a unitary drape as illustrated in FIG. 3, the drape needs to be correctly oriented when applying the drape over the robot for it to correctly fit the cart. Surgical drapes are typically applied to surgical robots (such as the one illustrated in FIG. 1) by first applying a part of the drape to the distal part of the arm, then unfolding or unfurling the remainder of the drape along the arm and over the cart. Thus the member of operating room (OR) staff applying the drape needs to correctly orient the cart part of the drape before applying the arm part of the drape to the robot arm. This is difficult and takes time. If the drape is not initially oriented correctly, it must be re-oriented partway through draping the robot. This can be awkward. As the drape unfurls, the side that comes into contact with the robot is then no longer sterile. This means that the drape cannot be rolled back up again for re-applying to the robot. Thus, in some situations the drape must be discarded and the process started again. This is wasteful, both of time and of materials.

The process of applying a drape can be further complicated by the requirement that the arm part of the drape correctly fits the robot arm. In the illustrated example, the arm part 302 is generally tubular (it might be generally cylindrical, generally or partly conical, or generally or partly frustoconical, at least to some degree, with an outward taper from a part of the drape for covering the distal end of the robot arm towards the base of the robot arm). The part of the drape for covering the distal end of the arm can have a particular orientation with respect to the arm. For instance, the drape may have a particular orientation with respect to the drive assembly interface of the distal end of the arm. Thus, this part of the drape will need to be in the correct orientation for it to be applied to the distal end of the arm.

The distal end of the arm can be controlled to be in different configurations relative to the remainder of the arm, for example by being rotated about a joint proximal of the distal end. Thus the distal end of the arm can be in different configurations relative to the cart to which the arm can be mounted. Therefore applying the drape to the distal end of the robot arm in the correct orientation, and then unfurling the drape along the arm, can mean that the drape is not ideally orientated relative to the cart. Further, the joints of the arm may be articulated such that the arm can adopt a variety of configurations.

The drape may therefore need to be twisted to correctly fit over one or both of the arm and cart. This complicates the process of applying the drape to the robot, and can mean that undesirable forces such as shear forces are applied to the drape.

In some situations, applying forces to the drape, such as by twisting the drape, can adversely affect the integrity of the drape. For example, the drape may rip or tear, or otherwise rupture, thus potentially compromising the sterile barrier. Such a rupture might occur at a portion which experiences shear forces. A rupture in the drape would risk exposing the sterile field to the non-sterile robot. The sterile barrier may additionally or alternatively be compromised by the drape being thinned, or worn away, over time as well as by the drape rupturing. It is therefore important to maintain the integrity of the drape to ensure that the sterile barrier is not compromised.

A force within the drape material may be transferred to a part of the robot, for example the part of the robot adjacent the drape material. This can impair the performance of the robot. For example, the force on the robot caused by the drape can restrict free movement of the robot. The force on the robot caused by the drape can affect force feedback measurements which might sense forces on one or more portion of the robot. Thus the forces caused by the drape might introduce inaccuracies in such force feedback measurements.

As mentioned, the robot comprises a plurality of joints. The robot may comprise one or more types of joint, for example different types of revolute joint. An example of a revolute joint is one which joins two parts of the robot such that the joint axis (about which the parts of the robot adjacent the joint rotate relative to one another) is not aligned with the extent of one or both of the adjacent parts of the robot. The joint axis may not be aligned with the direction of elongation of one or both of the adjacent parts of the robot. For example, the axis of rotation may be inclined (such as by being perpendicular) to a direction in which a part of the robot adjacent the joint extends. An example of such a revolute joint is illustrated at 122 in FIG. 1. The joint 122 enables relative movement between a distal arm segment 124 and a proximal arm segment 126. The longitudinal axes of the distal and proximal arm segments extend in the plane of the figure. The axis of rotation of the joint 122 extends out of the plane of the figure, i.e. perpendicular to the longitudinal (or axial) direction of the distal arm segment 124 and the proximal arm segment 126. This type of joint typically imposes a restriction on the rotation that the joint will allow. For example, the joint may permit rotation up to approximately 360 degrees. Attempting a greater rotation may cause the parts attached to the joint to come into contact with one another. For example, referring to FIG. 1, where the proximal arm segment 126 is held in a fixed position, the distal arm segment 124 is rotatable in an anticlockwise direction from a position in which it points generally downwards to a position in which it points generally to the left of the cart 108. The presence of the proximal arm segment 126 impedes further rotation. The rotation of such a joint can therefore be considered to be constrained in that the joint does not permit rotation indefinitely, or freely.

Another example of a revolute joint is one which joins two parts of the robot such that the joint axis is generally or substantially aligned with a direction in which a part of the robot adjacent that joint extends. The joint axis of such a joint can be along (or substantially along) a longitudinal (or axial) direction of a part of the robot such as a longitudinally-extending part, for example a portion of the arm. An example of such a joint is illustrated at 120 in FIG. 1. The joint 120 enables relative movement between a part of the robot distal of the joint (for example the base 102 of the robot arm) and a part of the robot proximal of the joint (for example an upper part 128 of the cart 108). The axial direction of the segment of the arm comprising the base 102 of the arm (i.e. that part which extends between the joint 120 and the next distal joint 130) extends in the plane of the figure. The axial direction of the join 120 also extends along the axial direction of the base 102 of the arm. Thus, the joint 120 is configured to permit the base 102 of the arm to rotate about its axial direction. The joint 120 permits the base 102 of the arm to undergo axial rotation. The joint 120 has a joint axis whereby an adjacent part of the robot, for example a part of the robot attached to the joint 120, is axially rotatable. This type of joint typically does not impose a restriction on the rotation that the joint will allow. For example, the joint may permit rotation up to and beyond 360 degrees. The joint may permit rotation of two or more multiples of 360 degrees, i.e. more than one complete turn. The rotation of such a joint can therefore be considered to be unconstrained in that the joint permits multiple turns. The joint can be said to permit free rotation. Thus parts joined by the joint can be freely rotatable relative to one another. An example of this latter type of revolute joint is a roll joint in a robot arm. Such a roll joint may be configured to rotate by up to a predefined angular amount, for example 720 degrees. This value can be chosen in dependence on the rotation tolerance of cabling, such as data/power cabling, within the arm. Such a joint may be considered to permit free rotation. Rotation of parts joined by such a joint can rotate without hindrance up to the predefined angular amount.

The drape may cover a joint, such as a roll joint, that enables free, or unconstrained, axial rotation of an arm segment. The rotation of the arm segment can cause the drape to become twisted about that joint. For example, as discussed, the robot arm can attach to the cart via a base joint 120 adjacent the base 102 of the arm. The base joint 120 permits the base of the robot arm to rotate about its longitudinal axis. Such a joint can permit unconstrained rotation. The base of the arm can rotate freely about its longitudinal axis. It can make a complete turn of 360 degrees, and can make as many complete turns as is desired. A rotation of any number of complete or partial turns may be possible.

Referring again to the example of the drape illustrated in FIG. 3, as the base 102 of the robot arm covered by the drape undergoes axial rotation about the joint 120 joining the arm and the cart, the drape will become twisted. The twisting of the drape will tend to concentrate at the area indicated by the arrow 306. It is likely that, as the drape twists, it will be put under an increasing force, which may eventually lead to rupture of the drape. To avoid this, restrictions can be placed on the rotation of the arm. For example, the control system may prevent the arm from rotating more than two complete turns in one direction. This restriction on the movement of the arm due to the drape is undesirable. Such a restriction may cause the arm to reposition from one position to another by taking a more complex path than would otherwise be necessary, so as to ensure that the drape does not become too twisted. Thus the drape can hinder the efficient operation of the robot. Limitations on the movement of the drape can hinder the efficient operation of the robot.

Such restrictions can be reduced or avoided by providing a surgical robot drape that enables different portions of the drape to be freely rotatable relative to one another. I.e. the different portions of the drape can undergo unconstrained rotation relative to one another. The different portions of the drape are suitably able to rotate relative to one another indefinitely.

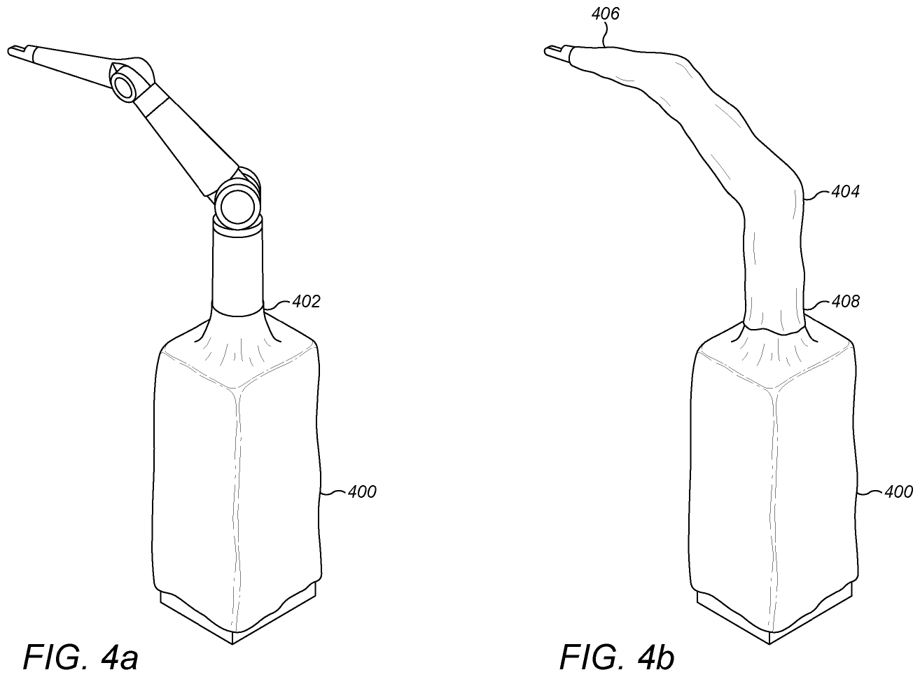

In one example, illustrated in FIGS. 4a and 4b, the surgical drape comprises a cart drape portion 400. The cart drape portion 400 is for covering the cart so as to provide a sterile barrier over the cart. The cart drape portion 400 is configured to be a close fit over the cart. This reduces the chances of the cart drape portion interfering in the location and/or movement of the robot during the surgical procedure. The cart drape portion is configured to be rotationally fast (or substantially rotationally fast) with the cart. For example, as illustrated, rotation of the cart drape portion relative to the cart (for example about a vertical axis of the cart) is restricted.

A drape (or drape portion) may be considered to be fast, or rotationally fast, with a part of the robot covered by the drape where the movement of the drape relative to the robot is restricted. For example, the drape may be a close fit about the part of the robot. Movement, such as rotational movement, may be restricted in at least one direction. The drape may be considered to be fast with the robot where the drape moves in registration with the robot. The drape may be fast with the robot where it is connected or attached to the robot.

The cart drape portion can be applied to the cart in one of four orientations. This is because, in the illustrated example, the cart is of a generally rectangular prism shape. In other examples, the cart drape portion may have a different number of orientations with respect to the cart. The cart drape portion is rotationally fast with the cart in the sense that turning the cart about its vertical axis will cause the cart drape portion to also turn about this axis. The cart drape portion will turn together with the cart. The cart drape portion will not substantially rotate relative to the cart itself. Similarly, where the cart is retained in a fixed orientation, the cart drape portion will also be substantially retained in that orientation. The cart drape portion need not be attached to the cart. Suitably the shape of the cart drape portion restricts relative movement between the cart drape portion and the cart. I.e. the cart drape portion can be configured to restrict relative movement between the cart drape portion and the cart. The cart drape portion 400 comprises a collar 402. The collar 402 is disposed at the top of the cart drape portion (in the cart orientation illustrated in FIG. 4). The collar 402 is arranged to extend past the top of the cart, so as to envelop a joint at the base of a robot arm mounted to the top of the cart. Suitably the collar 402 is configured to be a close fit about the circumference of the part of the robot arm extending from the cart. Suitably the collar 402 of the cart drape portion 400 is arranged to be a close fit about the circumference of the base 102 of the robot arm. The collar 402 is not attached to the robot arm. Hence, as the robot arm rotates about a joint such as a joint permitting axial rotation of the base 102 of the arm about a joint axis, it will rotate within the collar 402. Thus rotation of the arm will not cause the cart drape portion 400 to become twisted.

The drape further comprises a robot arm drape portion 404. The arm drape portion 404 is for covering the robot arm so as to provide a sterile barrier over the arm. The arm drape portion 404 is configured to be a close fit over the arm. This reduces the chances of the arm drape interfering in the location and/or movement of the robot arm and/or instruments during the surgical procedure. The arm drape portion comprises a distal part 406 for covering the distal part of the arm. The arm drape portion comprises a proximal part 408 for covering the proximal part of the arm. The proximal part 408 of the arm drape portion 404 is suitably arranged, in use, to envelop the base 102 of the arm. The arm drape portion is configured to be rotationally fast (or substantially rotationally fast) with the arm. For example, the arm is rotatable about a joint joining the arm and the cart. The arm is rotatable in this example about a vertical axis. In other examples, the axis of rotation need not be vertical (see, for example, FIG. 1, where the joint axis of the joint 120 joining the base 102 of the arm and the cart is at approximately 45 degrees to vertical). As the arm rotates about the joint axis, the arm drape portion enveloping the arm will move together with the arm. The material of the arm drape portion will generally move in registration with the arm covered by the arm drape portion. As the base 102 of the arm rotates about the joint axis of the joint 120 joining the arm and the cart, the proximal part 408 of the arm drape portion 404 will also rotate about the joint axis. Suitably the shape of the arm drape portion restricts relative movement between the arm drape portion and the arm. I.e. the arm drape portion can be configured to restrict relative movement between the arm drape portion and the arm.

Thus, as the arm rotates relative to the cart, the proximal part 408 of the arm drape portion 404 will rotate relative to the collar 402.

As can be seen from FIG. 4b, the arm drape portion 404 extends past the collar 402 of the cart drape portion 400. Thus the drape is configured such that there is an overlap in the material, such as the sheet material, of the drape. In the illustrated example, the overlap is adjacent the joint about which the arm can rotate relative to the cart. The overlap in the drape between the arm drape portion 404 and the cart drape portion 400 can provide a labyrinthine seal between the arm and cart drape portions, and so can assist in maintaining the sterile barrier provided by the drape. The arm drape portion can comprise a collar shroud for shrouding the collar of the cart drape portion. For example, the collar shroud can be provided at or towards an end of the arm drape portion that is arranged to be adjacent the cart drape portion when draping a robot.

Suitably the drape restricts or prevents the passage of fluid and/or particulate matter through the drape. Suitably, the overlap between the arm and cart drape portions restricts or prevents the passage of fluid and/or particulate matter between the arm drape portion and the cart drape portion. The sterile barrier provided by the drape can thereby be maintained.

The provision of an arm drape portion and a cart drape portion, which together can form a drape for covering a surgical robot to provide a sterile barrier thereover, enables the arm and cart to be separately draped by a member of OR staff. Since the arm drape portion and the cart drape portion can adopt different relative rotations relative to one another, the orientation of the arm need not affect the draping of the cart with the cart drape portion, and the orientation of the cart need not affect the draping of the arm with the arm drape portion. The arm and cart drape portions can be separately applied. The arm and cart drape portions can be joined together after having been applied over the respective parts of the robot. For example, the cart drape portion can be applied over the cart, an arm can be mounted onto the cart, for example by mounting the base of the arm through an aperture in the cart drape portion, and the arm drape portion can be applied over the arm, and joined to the cart drape portion. This approach can reduce the time and/or effort needed to appropriately drape the robot.

The overlap of the arm drape portion 404 and the cart drape portion 400 enables the two drape portions to be freely rotatable relative to one another. The two drape portions are freely rotatable relative to one another about the axis of the joint covered by the drape, for example the axis of the joint adjacent or enveloped by the overlapping drape portions.

One or both of the collar 402 and the proximal part 408 of the arm drape portion can be configured to restrict or prevent the passage of fluid and/or particulate matter through the drape. One or both of the collar 402 and the proximal part 408 of the arm drape portion, for example the collar shroud, can be configured to be a close fit about the covered portion of the robot, for example the base 102 of the robot arm. Suitably the collar shroud is configured to be a close fit about the collar. One or both of the collar 402 and the proximal part 408 of the arm drape portion can comprise a resilient portion. The resilient portion may be part of the drape material. The resilient portion may comprise a resilient element. The resilient element may be attached to the drape, for example to the arm drape portion and/or to the cart drape portion. The resilient portion is suitably configured to urge a circumferential extent of the respective part of the drape to adopt a relatively smaller circumference than when unconstrained, for example compared to where the resilient portion is not present. In this way, the drape can be configured such that one or both of the collar 402 and the proximal part 408 of the arm drape portion can be a close fit about a range of sizes of robot. Suitably the close fit of the collar and/or the proximal part of the arm drape portion is not so tight as to restrict the free rotation of the arm drape portion relative to the cart drape portion.

In some examples, the collar 402 can comprise a magnetic material, such as a magnet, for urging the collar towards another magnetic material. For example, where the base 102 of the arm is magnetic, the provision of a magnetic material on or forming part of the collar can cause the material of the collar to be urged towards the robot arm. This can help ensure a close fit of the collar about the arm. In some examples, the shroud can comprise a magnetic material, such as a magnet, for urging the shroud towards at least one of the collar and the arm covered by the drape. This can help ensure a close fit of the shroud about either or both of the collar and the arm. The strength of the magnetic material can be selected as desired. Suitably the strength of the magnetic material is such as to urge the collar towards the arm whilst permitting free rotation of the collar about the arm. Suitably the strength of the magnetic material is such as to urge the shroud towards one or both of the collar and the arm whilst permitting free rotation of the shroud about the collar.

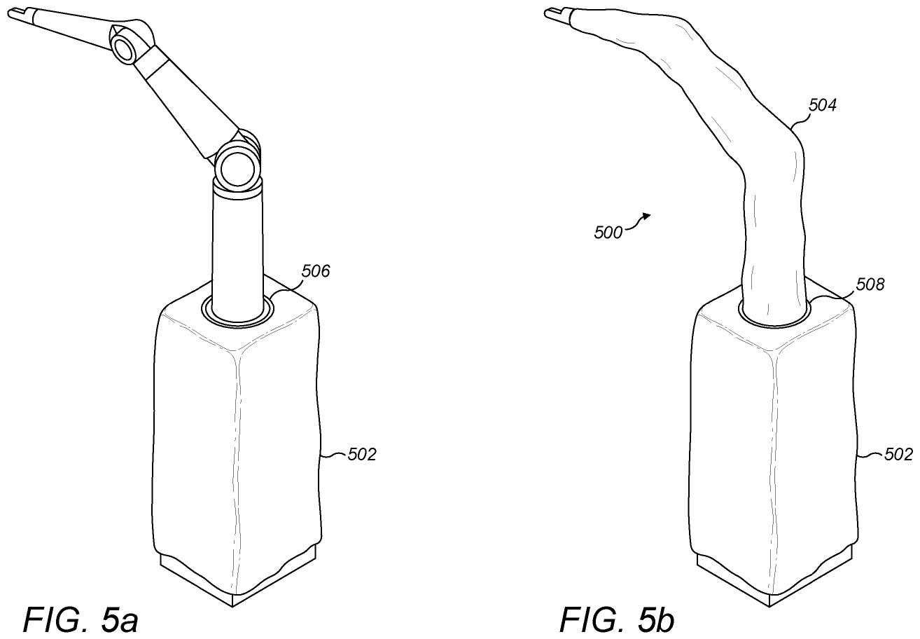

Another example of a surgical robot arm drape will now be described with reference to FIGS. 5a and 5b. The drape 500 comprises a cart drape portion 502 and an arm drape portion 504. FIG. 5a illustrates a robot having a cart and an arm, in which the cart drape portion has been applied over the cart. As discussed with reference to FIGS. 4a and 4b, the cart drape portion 502 is for covering the cart so as to provide a sterile barrier over the cart. The cart drape portion 502 is configured to be a close fit over the cart. Thus, similarly to the discussion regarding the example illustrated in FIGS. 4a and 4b, the cart drape portion 502 is configured to be substantially rotationally fast with the cart. The cart drape portion 502 comprises a connector 506 at an upper part of the cart drape portion. As illustrated, the connector 506 takes the form of a ring, or is ring-shaped. The connector 506 is for connecting the cart drape portion 502 to the arm drape portion 504. Suitably the cart drape portion 502 is configured such that the connector 506 is disposable about a joint of the robot. Suitably the connector 506 is disposable about a joint joining the arm and the cart, such as a roll joint.

As discussed with reference to FIGS. 4a and 4b, the arm drape portion 504 is for covering the robot arm so as to provide a sterile barrier over the arm. The arm drape portion 504 is configured to be a close fit over the arm. Thus, similarly to the discussion regarding the example illustrated in FIGS. 4a and 4b, the arm drape portion 504 is configured to be substantially rotationally fast with the arm. The arm drape portion 504 comprises a further connector 508 at a lower part of the arm drape portion. As illustrated, the further connector 508 takes the form of a ring, or is ring-shaped. The further connector 508 is for connecting the arm drape portion 504 to the cart drape portion 508. Suitably the arm drape portion 504 is configured such that the further connector 508 is disposable about a joint of the robot. Suitably the further connector 508 is disposable about a joint joining the arm and the cart, such as a roll joint.

The connector 506 of the cart drape portion 502 and the further connector 508 of the arm drape portion 504 are configured to connect to one another. The engagement between the connector 506 and the further connector 508 is suitably such as to restrict the passage of fluid and/or particulate matter between the arm and cart drape portions so as to maintain the sterile barrier provided by the drape. The connector 506 and the further connector 508 can be considered to form a coupling arrangement. The coupling arrangement is for coupling the two drape portions to one another. Suitably the coupling arrangement is configured to sealingly couple the two drape portions to one another.

Suitably, the connector 506 and the further connector 508, i.e. the coupling arrangement, are configured to permit rotation of the connector 506 relative to the further connector 508. Suitably the permitted rotation is unconstrained. For example, the connector 506 is suitably able to rotate an unlimited number of times relative to the further connector 508. Thus the connector 506 and the further connector 508 can freely rotate relative to one another. In this way, the coupling arrangement can be configured to permit unconstrained relative rotation between the cart and arm drape portions, of which the connector and the further connector form respective parts. The rotation between the connector 506 and the further connector 508 is suitably about an axis of at least one of the connector and the further connector. Suitably, the axes of the ring-shaped connector and ring-shaped further connector are aligned along a common axis, and the rotation is about this common axis.

The connector 506, for example a first coupling, and the further connector 508, for example a second coupling, are configured for snap-fit engagement with one another. The connector 506 may snap-fit into or around, or otherwise with, the further connector 508. One or both of the connector 506 and the further connector 508 comprises a resilient element to facilitate the snap-fit engagement.

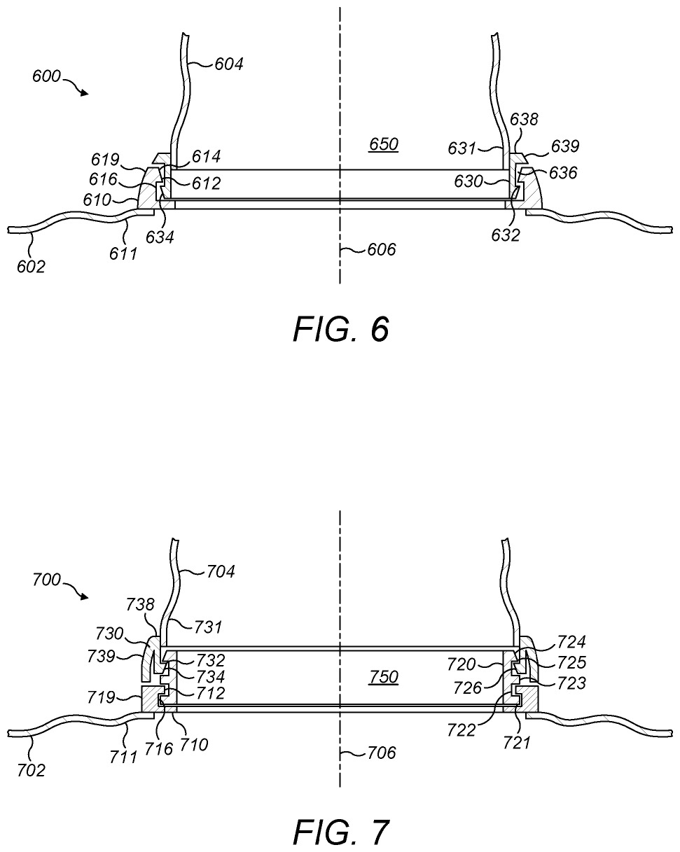

The following discussion will describe the coupling arrangement in more detail with reference to FIG. 6 and FIG. 7. FIGS. 6 and 7 show sectional views of the coupling arrangements. FIG. 6 illustrates a coupling arrangement 600 between a cart drape portion 602 and an arm drape portion 604. The dash-dotted line 606 shows the axis of the rings, i.e. the ring-shaped connector and the ring-shaped further connector.

The cart drape portion 602 comprises the connector 610. The connector is attached to the sheet material 611 of the cart drape portion. The arm drape portion 604 comprises the further connector 630. The further connector is attached to the sheet material 631 of the arm drape portion. The diameter of the further connector 630 is generally less than the diameter of the connector 610. This enables the further connector 630 to sit at least partially within the connector 610. This arrangement can help maintain the sterile barrier. In some examples, the connector can be receivable at least partially within the further connector. For example, the diameter of the connector 610 may be generally less than the diameter of the further connector 630.

The connector 610 comprises a first lip 612. The connector 610 comprises a first tapered part 614. The first lip 612 is provided at one end of the tapered part 614. The further connector 630 comprises a second lip 632. The further connector 630 comprises a second tapered part 634. The second lip 632 is provided at one end of the second tapered part 634. The first lip 612 and the second lip 632 form projections. The connector 610 comprises a first recess 616. The second lip 632 is receivable into the first recess 616. The further connector 630 comprises a second recess 636. The first lip 612 is receivable into the second recess 636.

In the configuration shown in FIG. 6, the first lip of the connector is within the second recess of the further connector, and the second lip of the further connector is within the first recess of the connector. This arrangement permits the connector and the further connector to engage with one another. It will be understood that it is not necessary in all examples for the first lip to be in the second recess and for the second lip to be in the first recess. It will be sufficient for one respective lip to be in the respective recess. The provision of the additional lip and recess can provide an enhanced engagement between the arm and cart drape portions. The additional lip and recess can provide an enhanced seal between the arm and cart drape portions.

Suitably at least one of the connector and further connector comprise a resilient portion. The resilient portion is suitably configured to permit deflection of the respective lip 612, 632 so that it may pass more easily into the respective recess 636, 616. This permits the snap-fit engagement between the connector and the further connector. Once the respective lip is within the respective recess, the resilience of the resilient portion enables the respective lip to return to its undeflected configuration, and to retain the connector and the further connector in an engaged arrangement. For example, the tapered parts 614, 634 can move past one another so as to cause engagement between the connector and the further connector. Only one of the connector and the further connector need comprise a tapered part. The respective tapered part can cause deflection of the respective lip provided at the end of that tapered part.

The first recess 616 is an annular recess. The first recess 616 extends about the circumference of the connector 610. The second recess 636 is an annular recess. The second recess 636 extends about the circumference of the further connector 630. The first lip 612 may comprise more than one projection for projecting into the second recess. The second lip 632 may comprise more than one projection for projecting into the first recess. Suitably, where a plurality of projections are provided, the projections are spaced, for example equally spaced, about the circumference of the connector or further connector, respectively. This permits a good engagement between the connector and the further connector about the circumference of the connectors.

The projection, such as the first lip, is able to move freely within the annular recess, such as the second recess. The annular recess defines a groove along which the projection can move. There is no limit to the number of times that the projection can pass around the annular recess. This means that the relative rotation between the connector and the further connector (and hence between the cart drape portion and the arm drape portion) is unconstrained. Such unconstrained relative rotation can be achieved whilst maintaining the engagement between the cart drape portion and the arm drape portion, and hence maintaining the sterile barrier.

The further connector 630, as illustrated in FIG. 6, comprises a flange, or rib, 638. The flange permits a user to press more easily on the further connector so as to urge it into engagement with the connector. A flange edge 639 of the flange 638 is configured to form a substantially continuous surface with an edge 619 of the connector. Such a substantially continuous or smooth outer profile of the coupling arrangement can help prevent fluid and/or particular matter from penetrating between the cart drape portion and the arm drape portion. It can also help prevent the coupling attachment from catching on materials in the OR.

The drape 602, 604 is configured to receive a robot, for example a joint of a robot, within a cavity 650 defined in the drape.

Referring to the coupling arrangement illustrated in FIG. 6, the first lip 612 is radially-inwardly facing and the second recess 636 is radially-outwardly facing. The second lip 632 is radially-outwardly facing and the first recess 616 is radially-inwardly facing. The coupling arrangement can be configured in some examples such that radially-inwardly facing elements are radially-outwardly facing, and vice versa.

The coupling arrangement permits the cart and arm drape portions to be applied over the respective parts of the robot in different relative orientations with respect to one another, and to be effectively joined at that orientation.

Another example of a coupling arrangement will now be discussed with reference to FIG. 7. FIG. 7 illustrates a coupling arrangement 700 between a cart drape portion 702 and an arm drape portion 704. The dash-dotted line 706 shows the axis of the rings, i.e. the ring-shaped connector and the ring-shaped further connector.

The cart drape portion 702 comprises the connector 710. The connector is attached to the sheet material 711 of the cart drape portion. The arm drape portion 704 comprises the further connector 730. The further connector is attached to the sheet material 731 of the arm drape portion.

In contrast with the example shown in FIG. 6, the coupling arrangement shown in FIG. 7 comprises an additional coupling element 720. The additional coupling element is disposed between the connector 710 and the further connector 730. The additional coupling element is configured to engage with both the connector and the further connector. In this way, the additional coupling element can join the connector and the further connector together. The additional coupling element 720 may be part of either the arm drape portion or the cart drape portion.

Referring to FIG. 7, the cart drape portion 702 comprises the additional coupling element 720. The connector 710 comprises a protrusion 712. The additional coupling element comprises a recess 722 defined between ribs (or projections) 721, 723. The protrusion 712 of the connector 710 is held within the recess 722. The recess is an annular recess, extending about the circumference of the additional coupling element. The connector 710 suitably comprises a plurality of protrusions 712. The protrusions 712 suitably extend about an inner circumference of the connector. The protrusions 712 are suitably spaced, for example equally spaced, about the circumference of the connector. This permits a good engagement between the connector and the further connector about the circumference of the connectors. The protrusion 712 is able to move freely within the recess 722. The recess 722 defines a groove along which the protrusion 712 can move. There is no limit to the number of times that the protrusion can pass around the recess. This means that the relative rotation between the connector and the additional coupling element is unconstrained. Such unconstrained relative rotation can be achieved whilst maintaining the sterile barrier.

The diameter of the further connector 730 is generally greater than the diameter of the additional coupling element 720. This enables the additional coupling element 720 to sit at least partially within the further connector 730. This arrangement can help maintain the sterile barrier. In some examples, the further connector can be receivable at least partially within the additional coupling element. For example, the diameter of the further connector 730 may be generally less than the diameter of the additional coupling element 720.

The further connector 730 comprises a third lip 732. The further connector comprises a third tapered part 734. The third lip 732 is provided at one end of the tapered part 734. The additional coupling element 720 comprises a fourth lip 725. The additional coupling element comprises a fourth tapered part 724. The fourth lip 725 is provided at one end of the fourth tapered part 724. The third lip 732 and the fourth lip 724 form projections. The third lip 732 and the fourth lip 724 are arranged, as shown in FIG. 7, to engage with one another so as to restrict the separation of the further connector 730 and the additional coupling element 720. The additional coupling element 720 comprises a fourth recess 726. The fourth recess 726 is defined between the rib 723 and the fourth lip 725. The third lip 732 is receivable into the fourth recess 726.

In the configuration shown in FIG. 7, the lip of the further connector (i.e. the third lip) is within the recess of the additional coupling element (i.e. the fourth recess). This arrangement permits the further connector and the additional coupling element to engage with one another.

Suitably at least one of the further connector and the additional coupling element comprise a resilient portion. The resilient portion is suitably configured to permit deflection of the respective lip 732, 725 so that it may pass more easily past the other lip, and/or so that lip 732 may pass more easily into recess 726. This permits the snap-fit engagement between the further connector and the additional coupling element. Once the lips 732, 725 have passed one another, and lip 732 is within the recess 726, the resilience of the resilient portion enables the lips 732, 725 to return to their undeflected configurations, and to retain the further connector and the additional coupling element in an engaged arrangement. For example, the tapered parts 734, 724 can move past one another so as to cause engagement between the further connector and the additional coupling element. Only one of the further connector and the additional coupling element need comprise a tapered part. The respective tapered part can cause deflection of the respective lip provided at the end of that tapered part.

The fourth recess 726 is an annular recess. The recess 726 extends about the circumference of the additional coupling element 720. The third lip 732 may comprise more than one projection for projecting into the fourth recess. Suitably, where a plurality of projections are provided, the projections are spaced, for example equally spaced, about the circumference of the further connector. This permits a good engagement between the further connector and the additional coupling element about their circumference.

The projection, such as the third lip 732, may be able to move freely within (for example along the annular extent of) the annular recess 726. The annular recess 726 defines a groove along which the projection 732 can move. There is no limit to the number of times that the projection 732 can pass around the recess 726. This means that the relative rotation between the further connector and the additional coupling element is unconstrained. Such unconstrained relative rotation can be achieved whilst maintaining the sterile barrier.

The further connector 730, as illustrated in FIG. 7, comprises a flange, or rib, 738. The flange permits a user to press more easily on the further connector so as to urge it into engagement with the additional coupling element. A flange edge 739 of the flange 738 is configured to form a substantially continuous surface with an edge 719 of the connector. Such a substantially continuous or smooth outer profile of the coupling arrangement can help prevent fluid and/or particulate matter from penetrating between the cart drape portion and the arm drape portion. It can also help prevent the coupling attachment from catching on materials in the OR.

The drape 702, 704 is configured to receive a robot, for example a joint of a robot, within a cavity 750 defined in the drape.

Referring to FIG. 7, the third lip 732 is radially-inwardly facing and the fourth recess 726 is radially-outwardly facing. The coupling arrangement can be configured in some examples such that radially-inwardly facing elements are radially-outwardly facing, and vice versa.

The provision of the additional coupling element can permit a consistency of rotational join between the arm and cart drape portions. For example, where, as illustrated in FIG. 7, the additional coupling element 720 is provided as part of the cart drape portion, the additional coupling element and the connector can be constructed so as to engage with one another in a manner which permits unconstrained rotation between them whilst forming a good seal. The engagement of the further connector of the arm drape portion with the additional coupling element need only form a good seal, and need not also provide a rotational join (although it might, as discussed herein). Thus the further connector and the part of the additional coupling element for coupling with the further connector can be configured to optimise the seal formed between them. In some examples, the further connector can engage with the additional coupling element so as to be fast with the additional coupling element. The rotation provided by the coupling arrangement can be provided by the relative rotation between the additional coupling element and the connector.

In some examples the coupling arrangement comprises portions of magnetic material. The provision of portions of magnetic material can aid the location of the parts of the coupling arrangement to one another so as to couple them together. For instance, the connector can comprise a magnet and the further connector can comprise a magnet. The connector magnet and the further connector magnet are suitably configured to attract one another so as to attract the connector and the further connector together. In some examples, the additional coupling element may comprise a magnet, and the further connector may comprise a magnet. The additional coupling element magnet and the further connector magnet are suitably configured to attract one another so as to attract the additional coupling element and the further connector together. The provision of magnets on the coupling arrangement can make it easier for a member of OR staff to join respective parts of the coupling arrangement to one another.

In some examples, a drape can accommodate flexion of a joint causing bending of a robot arm. The drape may accommodate relative movement between parts of the robot, for example the robot arm relative to the cart. A portion of drape material, or the configuration of a portion of drape material, can permit the drape to accommodate movement between parts of a robot covered by the drape. The following examples may be used in conjunction with any of the examples above, in any combination. They are described herein separately for clarity.

Referring to FIG. 8, a surgical robot arm drape 800 is provided which is arranged for covering a robot arm so as to provide a sterile barrier over the arm. The robot arm drape may be coupled to (for example by being unitary with) a cart drape portion. The drape 800 comprises a number of different regions along its length. The illustrated drape 800 comprises regions which are better able to accommodate flexing or bending of the drape 804, 808, 812 and regions which are less able to accommodate bending of the drape 802, 806, 810, 814. Towards the distal end of the drape is provided a first region 802. Adjacent the first region is a second region 804. To the other side of the second region from the first region is a third region 806. The second region separates the first and third regions from one another. To the other side of the third region from the second region is a fourth region 808. Adjacent the fourth region is a fifth region 810, followed by a sixth region 812 and a seventh region 814. Each of the regions may be formed separately and joined together. The regions may be joined by a coupling arrangement as discussed herein. Two of the regions may be formed unitarily and coupled to at least one other region, for example via a coupling arrangement discussed herein. Suitably a plurality of the regions are unitarily formed. For example, the first, second and third regions may be unitarily formed. This can simplify the draping of a robot using the drape.

The discussion herein will focus on the first, second and third regions. Regions 802, 806, 810 and 814 are similar to one another. Regions 804, 808 and 812 are similar to one another. Thus, the discussion of the first, second and third regions is also applicable to the third, fourth and fifth regions and the fifth, sixth and seventh regions.

The second region 804 is configured to be able to accommodate a greater change in its configuration (i.e. in the configuration of the second region) than the first and third regions. The second region is suitably able to accommodate the change in its configuration under a force, for example a force external to the drape. Such a force may be provided by a user handling the drape and/or by a robot arm enveloped by the drape undergoing movement such as driven movement. The second region is suitably preferentially deformed under an applied force. I.e. the second region is suitably more easily deformable than the first and third regions. The deformation of the second region can permit the accommodation of bending or flexing in the drape. Suitably the second region can accommodate bending of the drape without the sterile barrier thereby being compromised. Thus, in one example, bending movement (including repeated bending movement) can occur without causing a rip, tear and/or hole (or other rupture) in the drape.

The drape also suitably needs to allow articulation of the robot arm covered by the drape, such as in all of the degrees of freedom of the arm. For example, the drape may need to allow articulation in up to seven degrees of freedom. The drape should permit such articulation without transferring a force, in particular an unpredictable force, onto the arm. This is because where the drape exerts a force on the arm, this can lead to inaccurate force sensing by the arm.

The second region can comprise a compliant portion. The compliant portion is configured to accommodate movement such as bending of the drape without the drape thereby becoming taut. Thus the likelihood that the sterile barrier provided by the drape will be compromised is reduced. This can ensure that the sterile barrier is maintained throughout a surgical procedure.

In one example, as illustrated in FIG. 8, the second region 804 comprises sufficient material such that when the drape covers an arm, the material of the second region is baggy and/or unconstrained. The baggy, loose or unconstrained material is suitably sufficient to allow for the full range of motion of the arm or joint without the drape material stretching (or stretching beyond a resilient limit), ripping and/or tearing. The material of the second region is suitably less constrained than the material of the first and third regions. For example, the drape can comprise a plurality of fixing arrangements. The fixing arrangements can be for releasably fixing the drape to the arm. For example, the fixing arrangements can comprise one or more of ties for tying about the robot arm, magnets for attaching to a magnetic material of the arm, and so on. Suitably the fixing arrangements are disposed at or near the ends of the second region. For example, one fixing arrangement can be disposed at the boundary between the first region and the second region, or towards one side or the other of the boundary. Another fixing arrangement can be disposed at the boundary between the second region and the third region, or towards one side or the other of the boundary. The fixing arrangements can therefore allow the second region of the drape to be attached to a robot arm at a desired location, such as about a joint of the arm. The length (i.e. the longitudinal extent) of material of the second region can be greater than that needed to cover the joint. The excess material can be scrunched up so as to provide the relatively unconstrained material in the second region (i.e. about the joint). The second region can be retained in the scrunched-up configuration by means of the attachment of the fixing arrangements. Suitably, where a plurality of relatively unconstrained regions are desired, as illustrated in FIG. 8, a corresponding plurality of fixing arrangements can be provided. The relatively unconstrained material of the second region is disposed between the relatively more constrained material of the first and third regions.

The second region, and the first and third regions can, in one example, comprise different materials or materials with different properties or characteristics. The first and third regions suitably comprise the same material, or materials with the same properties or characteristics. Suitably the second region comprises a material having a lower modulus than the first and third regions. Suitably the second region has a configuration with a lower effective modulus than that of the first and third regions. The effective modulus is suitably the overall modulus of the region, for example the modulus due to the material and/or the configuration of that region. The material and the configuration of the first region need not both be different from the material and the configuration of the second region, although they might be. The material and the configuration of the third region need not both be different from the material and the configuration of the second region, although they might be. Suitably the second region comprises a material with a low modulus (for example an elastic or tensile modulus) or with a low friction coefficient, in at least one direction. The second region may comprise a material with a low modulus or with a low friction coefficient in a plurality of directions. A direction in which the second region has a low modulus and/or a low friction coefficient is suitably a bending (or flexing) direction of the drape, for example a direction along the longitudinal extent of the enclosure. A direction in which the second region has a low modulus and/or a low friction coefficient is suitably a twisting direction of the drape, for example a direction about a circumference of the enclosure or along a helical path along the enclosure.

In some examples, a modulus or friction coefficient can be considered to be low if the value for the second region is lower than the value for the first and third regions. In some examples, a modulus or friction coefficient can be considered to be low if the value for the second region is lower than a predetermined threshold value. Suitably the second region is of a material configured to deform under tension. The second region suitably comprises a material more easily deformed under tension than the material of the first and third regions. For example, the second region is of a material configured to stretch and/or shear under tension.

The second region can be formed from a resilient material, such as an elasticated material. The resilient material can comprise a material from the following group: an elastic, a rubber, a natural rubber, polyisoprene, an elastomer, polybutadiene, polychloroprene, butyl rubber, nitrile rubber, silicone rubber, a vinyl, and polyvinyl chloride. The resilient material can comprise a polymer such as a styrenic block copolymer. The styrenic block copolymer can comprise polystyrene blocks and rubber blocks. The rubber blocks can comprise polybutadiene, polyisoprene or hydrogenated equivalents thereof. This can permit stretching in the material without the material thereby rupturing. Suitably the resilience or elasticity of the second region is greater than that of the first region and of the third region. In some examples, the material of the second region is thinner than the material of the first and third regions. The thinner material of the second region can permit the material to have a greater compliance (for example to have a higher resilience and/or elasticity) than the relatively thicker material of the first and third regions.

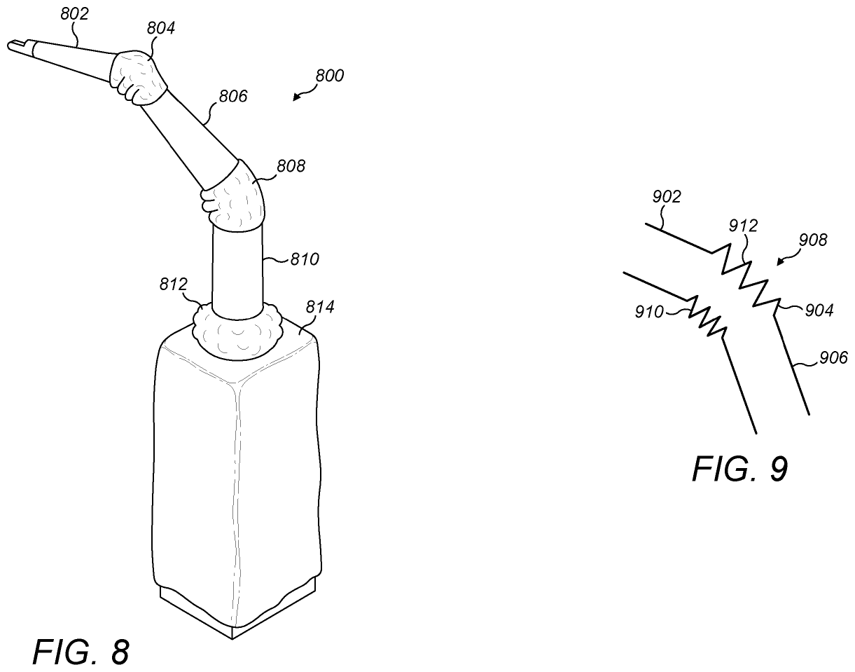

In another example, illustrated in FIG. 9, the second region 904 is again disposed between first 902 and third 906 regions. The second region comprises a ruckle (or folding/wrinkling) control portion, or a material control portion, for controlling the material in the second region. The ruckle control portion reduces the likelihood that the material of the second region ruckles (or folds/wrinkles) and/or controls the extent to which the material of the second region ruckles. The ruckle control portion is arranged to control the manner in which material of the second region moves as the drape flexes.

The ruckle control portion comprises a concertina section 908 in the material of the second region. The concertina section comprises a series of folds of material. The folds are at least one of pre-folded in the material and pre-stressed lines in the material. The concertina section has a contracted state in which the folds of material are relatively closer together, and an expanded state in which the folds of material are relatively further apart. As material, or part of the material, of the concertina section is put under increased tension the folds open up to permit movement of the material so as to restrict an increase in tension in the material. As material of a concertina section is put under reduced tension the folds close up to permit movement of the material so as to restrict a decrease in tension in the material. The folds of the concertina section may be arranged to close up on a decrease in tension under a resilience of the material from which the folds are formed. Tension in the drape material, for example in the enclosure formed by the drape, and in particular in the second region of the drape, can be changed as the drape bends. For example, as the generally tubular enclosure bends together with a robot arm enveloped by the drape one side of the drape will experience increased tension and the other side of the drape will experience reduced tension. A portion of the second region towards the outside of the bending joint will be in the expanded state 912. A portion of the second region towards the inside of the bending joint will be in the contracted state 910.

The concertina section can have an effective modulus (for example an elastic or tensile modulus) that is lower than that of the material from which it is formed, i.e. the material of the second region. Forming the material into folds can reduce the overall effective modulus of the section. As a result, the concertina section is suitably expandable under a lower force than that required to expand a flat section of material from which the concertina section is formed. Thus, as the enclosure is put under tensile load, the second region (comprising the concertina section) will preferentially deform. The second region will deform under a lower force than the first and third regions.

The folds of the concertina section suitably extend about the enclosure. The enclosure is suitably generally tubular, and the folds extend about the circumference of the generally tubular enclosure. The folds need not extend all the way round the enclosure.

The concertina sections described above can accommodate bending movement, such as movement about an arc. Bending movement will occur on the flexure of the robot arm. It is also possible to accommodate movement other than bending movement. For example, twisting movement can occur due to axial rotation about, for example, a roll joint. Suitably the second region is configured to accommodate bending and/or twisting movement. The folds or pleats of the concertina section are, in the example illustrated in FIG. 9 aligned circumferentially about at least part of the enclosure. For example, the folds can be linear folds in a sheet material that can be formed into the enclosure. In some examples, the folds can be provided in the material of the second region such that the direction of the folds in the enclosure is offset from a circumferential direction about the enclosure. For example, the folds can be arranged to adopt a helical configuration about the enclosure. The folds need not be provided about the whole of the circumference of the enclosure.

The second region may comprise two or more discontinuous concertina sections. Two of the concertina sections may differ from one another. The two concertina sections may differ from one another in one or more of: fold spacing, extent of the fold about the enclosure, angle the fold makes to the circumferential direction about the enclosure, number of folds.

Suitably the drape comprising the first, second and third regions is configured to be applied to a robot arm of a surgical robot. The robot arm can have a joint, and the drape can be configured to be applied over the arm such that the joint is enveloped by the second region. Suitably, the first region at least partially envelops a portion of the arm to one side of the joint and the third region at least partially envelops a portion of the arm to the other side of the joint. As the robot arm is controlled to move, the arm can flex about the joint. The drape is configured to be able to accommodate the flexion of the joint. Suitably, the drape accommodates the flexion of the joint without the material of the drape thereby becoming taut. This can reduce or avoid the drape thereby rupturing. This can help to maintain the sterile barrier. Suitably the second region is able to accommodate the flexion of the joint. Suitably the second region is better able to accommodate the flexion of the joint than the first region. Suitably the second region is better able to accommodate the flexion of the joint than the third region. The second region may be configured to accommodate the flexion of the joint by permitting a greater relative movement of the joint than the first and third regions, and/or by permitting a given relative movement of the joint to thereby cause a lower increase in material tension than the first and third regions.

The drape described herein could be used for non-surgical robotic purposes. For example, it could be used in robotic systems, or systems more generally, in which it is desirable to provide a barrier which accommodates relative motion of parts of the barrier. Such a barrier might be a barrier to fluid flow and/or a barrier to particulate matter, for example particulate matter entrained in a flow of fluid such as air. Such a barrier can therefore be used to provide effective protection from chemicals, material filings and/or dust.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A surgical robot drape for enveloping a joint of a surgical robot, the joint configured to permit axial rotation of a part of a robot arm adjacent the joint about a joint axis, the drape comprising:
a proximal drape material portion configured to provide a sterile barrier over, and to be rotationally fast with, a part of the robot proximal of the joint, and
a distal drape material portion configured to provide a sterile barrier over, and to be rotationally fast with, a part of the robot distal of the joint;
the drape being configured such that the proximal and distal drape material portions are freely rotatable relative to one another about the joint axis; and
the drape comprising a coupling arrangement for coupling the proximal drape material portion to the distal drape material portion, the coupling arrangement comprising a first coupling and a second coupling configured to couple to one another, wherein one of the proximal drape material portion and the distal drape material portion comprises the first coupling and the other of the proximal drape material portion and the distal drape material portion comprises the second coupling.

2. A surgical robot drape according to claim 1, in which the drape is configured to restrict the passage of fluid and/or particulate matter between the proximal and distal drape material portions so as to maintain the sterile barrier.

3. A surgical robot drape according to claim 1, in which the coupling arrangement is configured to sealingly couple the proximal and distal drape material portions.

4. A surgical robot drape according to claim 1, in which the coupling arrangement is configured to permit unconstrained relative rotation between the proximal and distal drape material portions.

5. A surgical robot drape according to claim 1, in which the first coupling and the second coupling are configured to engage with one another by a snap-fit engagement.

6. A surgical robot drape according to claim 1, in which one of the first and second couplings comprises a recess and the other of the first and second couplings comprises a protrusion, the recess being configured to receive the protrusion therein.

7. A surgical robot drape according to claim 6, in which the protrusion is configured to snap-fit past one of a lip and a rib adjacent the recess.

8. A surgical robot drape according to claim 6, in which one or more of the protrusion, the lip and the rib is resiliently deformable.

9. A surgical robot drape according to claim 1, in which the first coupling comprises a third coupling, and the first coupling is configured to couple to the second coupling via the third coupling.

10. A surgical robot drape according to claim 9, in which the third coupling is freely rotatable relative to the second coupling.

11. A surgical robot drape according to claim 9, in which the coupling arrangement is configured so that the second coupling and the third coupling are configured to engage with one another by a snap-fit engagement.

12. A surgical robot drape according to claim 9, in which the third coupling comprises one of a recess and a protrusion for engagement with the protrusion or recess of the second coupling.

13. A surgical robot drape according to claim 1, in which one of the proximal drape material portion and the distal drape material portion comprises a collar and the other of the proximal drape material portion and the distal drape material portion comprises a collar shroud.

14. A surgical robot drape according to claim 13, in which the collar shroud is configured to be a close fit about the collar.

15. A surgical robot drape according to claim 13, in which one or both of the collar and the collar shroud comprises a resilient portion.

16. A surgical robot drape according to claim 15, in which the material of the respective drape material portion comprises the resilient portion.

17. A surgical robot drape according to claim 15, in which the resilient portion is configured to urge a circumferential extent of the respective drape material portion to adopt a relatively smaller circumference than when unconstrained.

18. A surgical robot drape according to claim 1, in which at least one of the proximal drape material portion and the distal drape material portion comprises a magnetic material.

19. A surgical robotic system comprising a surgical robot and a surgical robot drape according to claim 1, in which the surgical robot drape is disposed about the surgical robot so as to envelop a joint of the surgical robot.

20.-40. (canceled)
